SEQUENCE LISTING (5 sequences)

SEQ ID NO: 1 - protein, 7 aa, Ganoderma microsporum
TLAWNVK

SEQ ID NO: 2 - protein, 14 aa, Ganoderma microsporum
PNWGRGRPSSFIDT

SEQ ID NO: 3 - protein, 11 aa, Ganoderma microsporum
YNSGYGIADTN

SEQ ID NO: 4 - protein, 111 aa, Ganoderma microsporum
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 5 - protein, 140 aa, synthetic construct
EAEAEFMSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWNYLEQKLISEEDLNSAVDHHHHHH

(19) United States
(12) Patent Application Publication
(10) Pub. No.: US 2024/0216465 A1
(43) Pub. Date: Jul. 4, 2024

(54) METHOD FOR TREATING REFRACTORY BRAIN TUMOR

(71) Applicant: National Yang Ming Chiao Tung University, Taiwan (TW)

(72) Inventors: Tung-Yi Lin, Taiwan (TW); Tsung-Hsi Tu, Taiwan (TW); Ai-Jung Tseng, Taiwan (TW)

(21) Appl. No.: 18/150,058

(22) Filed: Jan. 4, 2023

(51) Int. Cl. A61K 38/16; A61K 31/495; A61K 36/074; A61K 38/08; A61P 25/00; A61P 35/00

(52) U.S. Cl. CPC A61K 38/16; A61K 31/495; A61K 36/074; A61K 38/08; A61P 25/00; A61P 35/00

(57) ABSTRACT

Provided is a method for preventing and treating brain tumor, especially refractory brain tumor, glioblastoma multiforme (GBM), or refractory GBM in a subject in need thereof, including administering to the subject with an effective amount of an immunomodulatory protein of Ganoderma, a recombinant thereof, or a fungal immunomodulatory protein of a similar structure.

### SEQUENCE LISTING

Pursuant to 37 CFR § 1.831-835, the instant application contains a computer readable Sequence Listing which has been submitted electronically in XML format and is hereby incorporated by reference in its entirety. Said XML format file, created on Jan. 30, 2023, is named Sequence Listing.xml and is 5.12 kb in size.

### TECHNICAL FIELD

The present disclosure relates to a method for treating a brain tumor, and particularly to a method for treating a refractory brain tumor.

### BACKGROUND

A malignant brain tumor is a fast-growing cancer that spreads to other areas of the brain and spine. Generally, brain tumors are graded from I to IV, according to their behavior, such as how fast they grow and how likely they are to grow back after treatment.

Glioblastoma Multiforme (GBM), malignant Grade IV glioma, is the most common and highly lethal brain tumor that affects the central nervous system of humans and presents a poor prognosis. Currently, the clinical management of GBM patients mostly depends on using surgical resection combined with radiotherapy and/or chemotherapy. Specifically, adjuvant chemotherapy with anticancer agents combined with various treatments is a common therapeutic strategy. Unfortunately, even with combined treatment strategies, a low survival rate still presents in GBM patients due to the heterogeneous nature and several characteristics, such as overexpression of oncogenes and epigenetic silencing of DNA repair enzymes in GBM cells. Moreover, the poor prognosis may be due to highly local intra-brain metastasis of GBM. Therefore, targeting of GBM growth and mobility and identification of novel/effective strategies for GBM treatment are urgently needed.

Until now, there is no optimal therapeutic product directing to malignant brain tumor, GBM, or GBM refractory to anti-cancer agents (i.e., drug-resistant GBM). Therefore, there remains an unmet need to develop an effective pharmaceutic agent with safety and tolerability to treat or prevent such conditions.

### SUMMARY

In view of the foregoing, the present disclosure provides a method for treating brain tumor in a subject in need thereof, comprising administering an effective amount of an immunomodulatory protein derived from Ganoderma, a recombinant thereof, or a fungal immunomodulatory protein of a similar structure to the subject.

In some embodiments, the immunomodulatory protein or the recombinant thereof is derived from Ganoderma lucidum, Ganoderma tsugae, Ganoderma microsporum. Ganoderma applanatum. Ganoderma japonicum, Ganoderma astum, Ganoderma atrum, or Ganoderma sinensis. In other embodiments, the immunomodulatory protein is LZ-8 derived from Ganoderma lucidum, FIP-gts derived from Ganoderma tsugae, GMI derived from Ganoderma microsporum, FIP-gap derived from Ganoderma applanatum. FIP-gja derived from Ganoderma japonicum. FIP-gas derived from Ganoderma astum, FIP-gat derived from Ganoderma atrum, or FIP-gsi derived from Ganoderma sinensis or any recombinant thereof.

In some embodiments, the brain tumor is malignant brain tumor, refractory brain tumor, high-grade glioma, GBM, or refractory GBM. In other embodiments, the brain tumor is primary brain tumor or secondary brain tumor. In further embodiments, the brain tumor is localized brain tumor, regional brain tumor, advanced brain tumor, or metastatic brain tumor.

In some embodiments, the treatment of the brain tumor comprises suppressing expression or inducing degradation of hepatocyte growth factor receptor (HGFR), O⁶-methylguanine methyltrasferase (MGMT) or alkylpurine-DNA-N-glycosylase (APNG) in a brain tumor. In some embodiments, the treatment of the brain tumor comprises inducing proteasome-dependent Slug degradation in a brain tumor.

In at least one embodiment, the pharmaceutical composition is locally or systemically delivered to the subject, and the subject can be a mammal, for example, a human.

In at least one embodiment, the pharmaceutical composition is administered orally or rectally. In other embodiments, the pharmaceutical composition is administered parenterally.

In at least one embodiment, the pharmaceutical composition can be administered orally or rectally through an appropriate formulation with carriers and excipients to form tablets, pills, capsules, liquids, gels, syrups, slurries, suspensions and the like. In other embodiments, the pharmaceutical composition can be administered by an inhaler to the respiratory tract for local or systemic treatment of cancers.

In at least one embodiment, the parenteral administration is intravenous, drip, subcutaneous, intraperitoneal or intramuscular injection, or intrathecal or intraventricular administration.

In some further embodiments, the method of the present disclosure further comprises administering a second active agent. The second active agent is used with the immunomodulatory protein sequentially, concurrently or separately.

The present disclosure also provides a use of an immunomodulatory protein of Ganoderma, a recombinant thereof, or a fungal immunomodulatory protein of a similar structure in the manufacture of a medicament for treating brain tumor in a subject in need thereof, and the pharmaceutical composition includes a buprenorphine or a derivative thereof. The present disclosure further provides a pharmaceutical composition for use in the treatment of brain tumor in a subject in need thereof, and the pharmaceutical composition includes an immunomodulatory protein of Ganoderma, a recombinant thereof, or a fungal immunomodulatory protein of a similar structure, and a pharmaceutically acceptable carrier thereof

The present disclosure additionally provides a pharmaceutical composition when used in treating opioid addiction or pain, and the pharmaceutical composition includes a buprenorphine or a derivative thereof, a stabilizer, and a biocompatible solvent.

The present disclosure also provides an aqueous formulation for parenteral administration comprising the said immunomodulatory protein as an active agent. In some embodiments, the amount of the active agent in the formulation ranges from about 0.5 mg/mL to about 150 mg/mL.

### BRIEF DESCRIPTION OF THE DRAWINGS

The above and other objects, features and effects of the invention will become more readily appreciated by reference to the following description in conjunction with the accompanying drawings.

FIGS. 1A-1E show GMI inhibits cell viability in GBM cells. FIG. 1A reveals GBM8401, U87 and T98G cells were treated with various concentrations of GMI (0-1.2 μM) for 24-72 h. Each group of GMI-treated samples was normalized against an untreated control (24 h). FIG. 1B shows cells were treated with GMI (0-1.2 μM) for 7 days. FIGS. 1C to 1D show cells were exposed to GMI (0-1.2 μM) for 48 h, and then stained with PI and Annexin V-FITC analyzed by flow cytometry. The distributions of cell cycle and apoptosis on the tested cells were analyzed by FlowJo software. FIG. 1E shows cells were treated with GMI (0-0.9 μM) for 48 h. Western blot analysis was performed to determine the levels of apoptotic molecules, including PARP and caspase 3. Tubulin was used as the internal control.

FIGS. 2A-2K show GMI suppressed tumorigenesis in C6-bearing Rat. FIG. 2A shows C6 cells were treated with various concentrations of GMI (0-0.9 μM) for 48 h. Each group of GMI-treated samples was normalized against an untreated control. FIG. 2B shows C6 cells were exposed to GMI (0.6 μM) for 48 h, and then stained with PI and Annexin V-FITC analyzed by flow cytometry. FIG. 2C shows C6 cells were treated with various concentrations of GMI (0-0.6 μM) for 3h.

FIGS. 2D to 2H show continuous treatment of GMI in C6-bearing rat. FIG. 2D shows schematic design of the in vivo GMI continuous treatment experiments.

FIG. 2E shows the tumor volume in rats received GMI (0.6 mg/kg) via tail-vein injection on Day 13.

FIG. 2F indicates the images of brain tumors were detected by MRI on day 13.

FIG. 2G indicates immunohistochemical analysis of HGFR was performed in GBM tissues.

FIG. 2H reveals HGFR levels in tumor lesions were detected by Western blot.

FIGS. 2I to 2J show the C6 cells were implanted in the rats and treated with GMI (5 mg/rat) via tail-vein injection on Day 13. FIG. 2I shows immunohistochemical analysis of GMI was performed in GBM tissues. FIG. 2J shows GMI levels in non-tumor and tumor lesions were detected by Western blot.

FIG. 2K shows survival rate of rats continuously received GMI (0.6 mg/kg) via tail-vein injection.

FIGS. 3A-3D show GMI inhibits cell viability in GBM patient-derived cells. FIG. 3A show the information of two patients with GBM. FIG. 3B indicates two patient-derived cells (PDCs) were treated with various concentrations of GMI (0-0.3 μM) for 48 h. The cell viability was determined by MTT assay. Each group of GMI-treated samples was normalized against an untreated control. FIG. 3C shows PDCs were treated with GMI (0-0.6 μM) for 48 h, and then stained with PI and Annexin V-FITC analyzed by flow cytometry. FIG. 3D shows PDCs were treated with GMI (0-0.3 μM) for 3 h. Western blot analysis was performed to determine the levels of HGFR-related molecules. Tubulin was used as the internal control.

FIGS. 4A-4G show GMI-combined with a DNA alkylating agent, Temozolomide (TMZ), synergistically induces cell death in GBM8401 and T98G cells. FIG. 4A shows Cells were treated with various concentrations of GMI for 48 h, followed by incubation with TMZ (0-50 μM) for another 24 h. Cell viability was determined using the MTT assay. FIG. 4B indicates Cells co-treated with GMI (0.6 μM) and TMZ (12.5 and 25 μM) for 7 days. FIGS. 4C and 4E show cells were exposed to GMI (0.9 μM for GBM8401 and T98G; 0.6 μM for PDC-I) and TMZ (50 μM) for 48 h. FIG. 4D shows cells were co-treated with GMI (0.6 μM) and TMZ (50 μM) for 48 h. FIGS. 4F and 4G show cells were treated with GMI (0-1.2 μM) for 48 h. Western blot analysis was performed to determine the levels of APNG and MGMT. Tubulin was used as the internal control.

FIG. 5 shows GMI reduces expressions of APNG and MGMT in tumor lesions of C6-bearing rat receiving GMI. HGFR, APNG and MGMT levels in tumor lesions were detected by Western blot. Tubulin was used as the internal control.

FIGS. 6A-6C show combined treatment of GMI and TMZ enhances the synergistic cytotoxic effects in rat C6 cells. FIG. 6A reveals cells were treated with GMI (0-1.2 μM) for 48 h, followed by incubation with TMZ (0-100 μM) for another 24 h. Cell viability was determined using the MTT assay. Each GMI and TMZ co-treated group was normalized against each untreated control. FIG. 6B shows The CI and DRI values of GMI combined with TMZ in C6 cells evaluated by CompuSyn™ software. FIG. 6C shows cells were exposed to GMI (0.6 μM) and TMZ (50 μM) for 48 h, followed stained with PI and Annexin V-FITC analyzed by flow cytometry.

FIGS. 7A-7F show GMI combined with TMZ suppressed tumorigenesis in C6-bearing rats. FIG. 7A indicates Schematic design of the in vivo post combined therapy of GMI and TMZ in C6-bearing rats. The tumor volume in rat received GMI (0.6 mg/kg) and TMZ (30 mg/kg) via tail-vein injection. FIG. 7B shows the images of brain tumors were detected by MRI on Day 13. FIG. 7C indicates the tumor volume was measured; each bar represents the mean+SD. FIG. 7D shows rat weight was measured at the indicated times; each bar represents the mean+SD. FIGS. 7E and 7F show the AST and ALT (hepatic function: shown in FIG. 7E) as well as BUN and creatinine (renal function; shown in FIG. 7F) of rat sera were analyzed.

FIGS. 8A-8C show GMI inhibits the migration and invasion of GBM8401 cells. FIG. 8A indicates GBM8401 cells were cultured at 0, 24 and 48 h in serum-free medium with GMI (0-0.6 μM) to determine wound closure. Data (% wound closure) is expressed as a percentage of the control (0 h). FIGS. 8B and 8C reveal cell mobility was determined using a Transwell assay. The migration (show in FIG. 8B) and invasion (C) of GBM8401 cells is inhibited by GMI (0.6 and 1.2 μM) for 24 h.

FIGS. 9A-9C show GMI combined TMZ inhibits cell migration and invasion of GBM8401 cells. FIG. 9A indicates GBM8401 cells were cultured at 0, 24 and 48 h in serum-free medium with GMI (0.6 μM) and TMZ (50 μM) to determine wound closure. Data (% wound closure) is expressed as a percentage of the control (0 h). FIGS. 9B and 9C show cell mobility was determined using a Transwell assay. The migration (shown in FIG. 9B) and invasion (FIG. 9C) of GBM8401 cells is inhibited by GMI (0.6 μM) and TMZ (50 μM) for 24 h.

FIGS. 10A-10D show GMI induces Slug degradation in GBM cells. FIG. 10A shows the expression of Slug mRNA (SNAI2) in normal and primary GBM tissue in the TCGA data resource. FIG. 10B reveals cells were treated with GMI (0-0.6 μM) for 3 and 24 h. FIG. 10C shows GBM8401 and T98G cells were treated with GMI (0.6 μM) for 3 h. The mRNA level of Slug was measured using qPCR. FIG. 10D shows cells were pretreated with DMSO (vehicle control) and MG132 (10 μM) for 30 mins, followed by incubation with GMI (0.6 μM) for 3 h.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

As used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “comprising,” “including,” “having,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitation.

As used herein, the term “an effective amount” is the quantity of an active agent which achieves a clinical outcome when the compound is administered to a subject. For example, when an active agent of the disclosure is administered to a subject with a cancer, a “clinical outcome” includes reduction in tumor mass, reduction in metastasis, reduction in the severity of the symptoms associated with the cancer and/or increase in the longevity of the subject. The effective amount may vary, as recognized by those skilled in the art, depending on routes of administration, excipient usage, the possibility of co-usage with other therapeutic treatment, and the condition to be treated.

According to the disclosure, the terms “treatment,” “treating” and the like are used herein to generally mean obtaining a desired pharmacologic or physiologic effect. The effect may be prophylactic in terms of completely or partially preventing a condition, appearance, disease or symptom and/or may be therapeutic in terms of a partial or complete cure for a condition and/or adverse effect attributable to a condition or disease. The term “treatment” as used herein covers any treatment of a condition, disease or undesirable appearance in a mammal, e.g., a human, and includes: (a) preventing the disease (e.g., cancer), condition (pain) or appearance (e.g., visible tumors) from occurring in a subject which may be predisposed to it but has not yet been observed or diagnosed as having it: (b) inhibiting the disease, condition or symptom, i.e., causing regression of a condition or symptom; and (c) relieving the disease, condition or symptom, i.e., causing regression of a condition or symptom.

As used herein, the terms “patient” and “subject” are used interchangeably. The term “subject” means a human or an animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments of the present disclosure, the subject is a mammal, e.g., a primate such as a human.

As used herein, the term “administering” or “administration” refers to the placement of an active agent into a subject by a method or route which results in at least partial localization of the active agent at a desired site to produce a desired effect. The active agent described herein may be administered by any appropriate route known in the art. For example, the pharmaceutical composition of the present disclosure is administered to the subject by oral administration.

As used herein, the term “recombinant” may refer to the alteration of genetic material by human intervention. For example, recombinant may refer to the manipulation of DNA or RNA in a cell or virus or an expression vector by molecular biology (recombinant DNA technology) methods, including cloning and recombination. Recombinant may also refer to manipulation of DNA or RNA in a cell or virus by random or directed mutagenesis. A “recombinant” nucleic acid can be described with reference to how it differs from a naturally occurring counterpart (the “wild-type”). A recombinant protein may refer to a protein expressed by recombinant DNA technology. A recombinant protein has a similar amino acid sequence and maintains the same activity or function as its parental protein.

The numeral ranges used herein are inclusive and combinable, any numeral value that falls within the numeral scope herein could be taken as a maximum or minimum value to derive the sub-ranges therefrom. For example, it should be understood that the numeral range “0.5 mg/mL to about 150 mg/mL” comprises any sub-ranges between the minimum value of 0.5 mg/mL to the maximum value of 150 mg/mL, such as the sub-ranges from 0.5 mg/mL to 100 mg/mL, from 1.0 mg/mL to 150 mg/mL, from 10 mg/mL to 80 mg/mL and so on. In addition, a plurality of numeral values used herein can be optionally selected as maximum and minimum values to derive numerical ranges. For instance, the numerical ranges of 5% to 60%, 10% to 40%, and 20% to 60% can be derived from the numeral values of 5%, 10%, 20%, 40%, and 60%.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

Lingzhi, an herbal mushroom, used in traditional Chinese medicine for at least 2,000 years, is a species complex that encompasses several fungal species of the genus Ganoderma, most commonly Ganoderma lucidum, Ganoderma tsugae, and Ganoderma sichuanense, which are closely related. Many therapeutic effects have been reported of Lingzhi, such as immunomodulatory, anti-tumor, hepato-protective, antioxidant, and cholesterol-lowering effects (Jinn et al., 2006, Biosci. Biotechnol. Biochem., 70, 2627-2634). Most of these therapeutic effects are attributed to triterpenoids, polysaccharides, and glycoproteins (Boh et al., 2007, Biotechnol. Annu. Rev., 13, 265-301; Jinn et al., 2006, Biosci. Biotechnol. Biochem., 70, 2627-2634). A glycoprotein class in Lingzhi named fungal immunomodulatory proteins (FIPs) has recently been identified. So far, at least 5 FIPs have been isolated, i.e., LZ-8, (Ganoderma lucidum), FIP-gts (Ganoderma tsugae), FIP-gja (Ganoderma sinensis) and GMI (Ganoderma microsporum) (Ko et al., 1995, Eur. J. Biochem., 228, 244-249).

In at least one embodiment of the present application, the immunomodulatory protein of Ganoderma microsporum (GMI) may suppress the expression of tyrosine kinase (RTKs) that may be used as therapeutic targeting in GBM patients, such as hepatocyte growth factor receptor (HGFR), epidermal growth factor receptor (EGFR), platelet-derived growth factor receptor (PDGFR), and vascular endothelial growth factor receptor (VEGFR), but the present disclosure is not limited thereto. Activation of HGFR and its downstream signaling pathways, such as mTOR, PI3K/AKT, Ras/ERK signalings and epithelial-mesenchymal transition (EMT)-associated molecules, are correlated to cellular proliferation, invasion, stem cell-like characteristics and drug resistance of GBM cells, leading to poor prognosis for patients with GBM. In some embodiments, immunomodulatory protein of Ganoderma microsporum (GMI) is effective in suppressing HGFR expression of brain tumor cells and treating brain tumors. In some aspects, the GMI induces HGFR degradation in a brain tumor cell through the proteasomal degradation system. For example, the GMI activates apoptosis in GBM cells, thereby inducing HGFR degradation. The tumorigenesis in C6-bearing rat is suppressed by GMI. Also, GMI inhibits cell viability in GBM patient-derived cells. In addition, the combination of GMI and Temozolomide (TMZ) synergistically induces cell death in GBM8401 and T98G cells. GMI reduces expressions of APNG and MGMT in tumor lesions of C6-bearing rat receiving GMI. These findings suggest efficacy GMI and/or TMZ in brain tumor immunotherapy.

In one aspect, the method of the present disclosure for treating brain tumor, refractory brain tumor, high-grade glioma, GBM, or refractory GBM comprises suppression of tumor growth, progression, or recurrence. In another aspect, the method of the present disclosure for treating brain tumor comprises prevention of brain tumor development.

In one aspect, the Ganoderma immunomodulatory protein, a recombinant thereof, or a fungal immunomodulatory protein of a similar structure in combination with an anti-cancer drug(s) provides an effect in the treatment and/or prevention of brain tumor, refractory brain tumor, high-grade glioma, GBM, or refractory GBM. In another aspect, the present disclosure provides a pharmaceutical composition, which comprises a Ganoderma immunomodulatory protein, a recombinant thereof, or a fungal immunomodulatory protein of a similar structure, and an anti-cancer agent. The composition exhibits a synergistic effect in treating and/or preventing brain tumor, refractory brain tumor, high-grade glioma, GBM, or refractory GBM.

In one embodiment, the Ganoderma immunomodulatory protein, a recombinant thereof, or a fungal immunomodulatory protein of a similar structure is derived from Ganoderma lucidum. Ganoderma tsugae. Ganoderma microsporum. Ganoderma applanatum. Ganoderma japonicum. Ganoderma astum. Ganoderma atrum, or Ganoderma sinensis. For example, the immunomodulatory protein is LZ-8 derived from Ganoderma lucidum, FIP-gts derived from Ganoderma tsugae, GMI derived from Ganoderma microsporum, FIP-gap derived from Ganoderma applanatum, FIP-gja derived from Ganoderma japonicum, FIP-gas derived from Ganoderma astum, FIP-gat derived from Ganoderma atrum, or FIP-gsi derived from Ganoderma sinensis or a recombinant thereof. In some embodiments, the immunomodulatory protein is derived from Ganoderma microsporum (GMI) or Ganoderma lucidum (LZ-8).

According to this disclosure, the Ganoderma immunomodulatory protein (e.g., GMI), a recombinant thereof, or a fungal immunomodulatory protein of a similar structure has the amino acid sequence of: (1) TLAWNVK (SEQ ID NO: 1), (2) PNWGRGRPSSFIDT (SEQ ID NO: 2), and (3) YNSGYGIADTN (SEQ ID NO: 3), or the amino acid sequence of MSDTALIFTLAWNVKQLAFDYTPNWGRGRPSSFIDTVTFPTVLTDKAYTY RVVVSGKDLGVRPSYAVESDGSQKINFLEYNSGYGIADTNTIQVYVIDPD TGNNFIVAQWN (SEQ ID NO: 4), or

the amino acid sequence of

(SEQ ID NO: 5)
EAEAEFMSDTALIFTLAWNVKQLAFDYTPNWGRGRPSSFIDTVTFPTVL
TDKAYTYRVVVSGKDLGVRPSYAVESDGSQKINFLEYNSGYGIADTNTI
QVYVIDPDTGNNFIVAQWNYLEQKLISEEDLNSAVDHHHHHH.

In at least one embodiment, the pharmaceutical composition comprising GMI of the present disclosure can be used in combination with radiotherapy and/or chemotherapy. In some embodiments, the pharmaceutical composition can be used in combination with radiotherapy and/or chemotherapy.

In at least one embodiment, the pharmaceutical composition comprising GMI of the present disclosure can be combined with an anti-cancer agent for combination therapy in brain tumor. The Ganoderma immunomodulatory protein, the recombinant thereof, or the fungal immunomodulatory protein of a similar structure in the present disclosure can further be combined with an anti-cancer agent as a pharmaceutical composition. For example, the present disclosure may provide a pharmaceutical composition comprising Ganoderma immunomodulatory protein, a recombinant thereof, or a fungal immunomodulatory protein of a similar structure and an anti-cancer agent, and said pharmaceutical composition can treat and/or prevent brain tumor. The brain tumor may be refractory brain tumor, high-grade glioma, GBM, or refractory GBM.

In at least one embodiment, the composition of the present disclosure exhibits a synergistic efficacy.

In some embodiments, the effective amount of the active agent of the pharmaceutical composition is about 0.01 mg to about 10 mg protein per 70 kg body weight for a human. In some further embodiments, the effective amount of the active agent is about 1.0 mg to about 5 mg protein per 70 kg body weight for a human. In other embodiments, the effective amount of the active agent used in the method of the present disclosure is about 10 mg to about 100 mg protein per 70 kg human body weight, e.g., about 20 mg to about 80 mg protein per 70 kg body weight, about 20 mg to about 50 mg protein per 70 kg body weight, about 25 mg to about 50 mg protein per 70 kg body weight, about 30 mg to about 50 mg protein per 70 kg body weight, about 35 mg to about 50 mg protein per 70 kg body weight, or about 30 mg to about 40 mg protein per 70 kg body weight.

In some embodiments, the effective amount of the active agent of the pharmaceutical composition used in the method of the present disclosure is about 0.001 mg to about 0.1 mg protein per 1 kg body weight, e.g., about 0.0125 mg protein per 1 kg body weight, or about 0.05 mg protein per 1 kg body weight. In other embodiments of the present disclosure, the effective amount of the active agent of the pharmaceutical composition may be administered to a subject 1 to 4 times per day, 1 to 4 times per week, or 1 to 4 times per month. In further embodiments of the present disclosure, the effective amount of the active agent of the pharmaceutical composition may be administered to a subject every 1, 2, 3, 4, 5, 6, or 7 days.

In another aspect, the present disclosure provides an aqueous formulation for parenteral administration comprising a Ganoderma immunomodulatory protein, a recombinant thereof or a fungal immunomodulatory protein of a similar structure as an active agent. In at least one embodiment, the amount of the active agent in the formulation ranges from about 0.5 mg/mL to about 150 mg/mL, e.g., about 1.0 mg/mL to about 100 mg/mL, about 1.0 mg/mL to about 80 mg/mL, about 1.0 mg/mL to about 60 mg/mL, about 1.0 mg/mL to about 40 mg/mL, about 1.0 mg/mL to about 20 mg/mL, about 5 mg/mL to about 150 mg/mL, about 5 mg/mL to about 100 mg/mL, about 10 mg/mL to about 150 mg/mL, or about 10 mg/mL to about 100 mg/mL.

The parenteral formulations may be in unit dose form in ampoules, small volume parenteral (SVP) vials, large volume parenterals (LVPs), pre-filled syringes, small volume infusion or in multi-dose containers. The formulations are suspensions or solutions and may contain formulatory agents such as preserving, wetting, buffering, emulsifying or suspending, stabilizing and/or dispersing agents. Alternatively, the ratio, type, and varieties of the ingredients, active and inactive, are studied to reach an optimal balance, before use with a suitable vehicle, e.g., sterile, pyrogen-free water. Some embodiments are contemplated that are substantially free of buffers, stabilizers, and/or preservatives, while still preserving the formulation's chemical stability, pH value, and product sterility.

Parenteral formulations are typically aqueous solutions which may contain excipients such as salts, carbohydrates and buffering agents (e.g., at a pH of from 3 to 9.5). Additional embodiments are substantially buffer free.

In some embodiments, the parenteral formulation comprises a single dose pH adjusted solution having an effective amount of an active agent for treating a brain tumor: a tonicity agent for adjusting osmolality to about physiological osmolality; optional pH adjusting reagents: and sterile water for injection.

In at least one embodiment, the parenteral formulations contain a solution of an active agent in an aqueous solvent combined with pH adjusting agents and at least one isotonicity agent. A water-insoluble inert gas may be carefully bubbled through the solvent to remove oxygen from the medium. Optionally, the formulations contain at least one preservative and/or at least one solubility enhancing agent and/or at least one stabilizing agent. In some embodiments, the formulation is substantially free of stabilizing agents and preservatives.

Tonicity agents are sometimes present. The term “tonicity agent” refers to a pharmaceutically acceptable excipient that makes the solution compatible with blood. Suitable tonicity agents include glycerin, lactose, mannitol, dextrose, sodium chloride, sodium sulfate, sorbitol and the like. In some embodiments, tonicity agents include mannitol, sorbitol, lactose and sodium chloride and any combinations thereof. The tonicity agent is added to the injectable to achieve substantially physiological osmolality for injection.

Hypertonic and hypotonic solutions both present complications and undesirable effects when injected. The parenteral formulations described herein are isotonic to minimize or avoid such effects. Since osmolality is the measure of particles in a solution, every component added to the injectable affects the osmolality, and thus adjusting to a final osmolality is complicated. For example, when also adjusting the pH, as addition of the tonicity agent may affect pH, addition of the pH adjusting reagents will affect tonicity.

Optional pH adjusting reagents include acids and bases, such as dilute HCl and NaOH. An acid may be added to lower the pH, while the base is added to raise the pH. In some instances, one or both an acid and a base may be used. In some embodiments, the pH adjusting reagents are chosen to complement the tonicity agent to provide similar ions when in solution. For example, when NaCl is used as a tonicity agent, HCl and/or NaOH may be used as the pH adjusting reagents.

The tonicity agent, such as NaCl, is employed to achieve an isotonic solution. Isotonic solutions for injection have an osmolality roughly equivalent to physiological osmolality. Other concentrations of NaCl may result in either undesirable hypertonic or hypotonic solutions.

Additional components, such as active agents, excipients, diluents, buffers, preservatives, etc. may be employed, so long as the parenteral formulation remains isotonic and stable. Any suitable additional active agent could optionally be incorporated into the parenteral formulation.

In some embodiments, the method and formulation described herein optionally further comprises a second active agent. The second active agent may be used with the immunomodulatory protein of the present disclosure sequentially, concurrently or separately. In at least one embodiment, the second active agent include, but is not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cisplatin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypernycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithineklerriene; emitefur; epirubicin; epristeride; erlotinib; estramustine analogue; estrogen agonists; estrogen antagonists; etanidazole; etoposide phosphate; exemestane; fadrozole; fazarabine; fenretinide; filgrastim;

finasteride; flavopiridol; flezelastine; fluasterone; fludarabine;
fluorodaunorubicin hydrochloride; forfenimex; formestane; fostriecin; fotemustine; gadolinium texaphyrin; gallium nitrate; galocitabine; ganirelix; gefitinib; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; lapatinib; leinamycin; lenograstim; lentinan sulfate; leptolstatin; letrozole; leukemia inhibiting factor; leukocyte alpha interferon; leuprolide plus estrogen plus progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitors; mifepristone; miltefosine; mirimostim; mismatched double stranded RNA; mitoguazone; mitolactol; mitomycin analogues; mitonafide; mitotoxin fibroblast growth factor-saporin; mitoxantrone;
mofarotene; molgramostim; monoclonal antibody; human chorionic gonadotrophin; monophosphoryl lipid A plus mycobacterial cell wall skeleton; mopidamol; multiple drug resistance gene inhibitors; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone plus pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulators; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetran; ras farnesyl protein transferase inhibitors; ras inhibitors; ras-GAP inhibitor; demethylated retelliptine; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonerrnin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Additional anti-cancer drugs may be 5-fluorouracil and leucovorin.

In some embodiments, the anti-cancer agent of the present disclosure can be selected from the group consisting of cisplatin, gefitinib, lapatinib, erlotinib, and TMZ. In at least one embodiment, the anti-cancer agent is TMZ.

Without further elaboration, it is believed that one skilled in the art can utilize the present disclosure to its fullest extent on the basis of the preceding description. The following examples are, therefore, to be construed as merely illustrative and not a limitation of the scope of the present disclosure in any way.

As used herein, chemotherapy includes the treatment of disease by means of chemicals that destroy cancerous tissue (anticancer therapy) or that have a specific toxic effect upon the disease producing microorganisms (antibiotics). This treatment modality produces favorable outcome in various cancers and bacterial diseases. However, it also produces generalized toxic effects such as nausea, vomiting, diarrhea, fever, fatigue, skin discoloration, rigours, and the like, reduces immune response, increases oxidative stress, and hampers the quality of life, predominantly in cancers.

As used herein, resistant cancer or refractory cancer is defined as the cancer that does not respond to medical treatment. It may be resistant at the beginning of treatment, or it may become resistant during treatment.

As used herein, O⁶-methylguanine methyltrasferase (MGMT) and alkylpurine-DNA-N-glycosylase (APNG) are two key molecules which are correlated to TMZ-resistance.

In at least one embodiment of the present disclosure, the pharmaceutically acceptable carrier may be a filler, a binder, a preservative, a disintegrating agent, a lubricant, a suspending agent, a wetting agent, a flavoring agent, a thickening agent, an acid, a biocompatible solvent, a surfactant, a complexation agent, or any combination thereof.

In at least one embodiment of the present disclosure, the pharmaceutical composition may be formulated into a form suitable for parenteral administration, injection, continuous perfusion, sublingual administration, subcutaneous administration, topical administration, or oral administration. For example, the pharmaceutical composition may be, but is not limited to, a formulation to injection, dry powder, a tablet, an oral liquid, a wafer, a film, a lozenge, a capsule, a granule, a pill, a gel, a lotion, an ointment, an emulsifier, a paste, a cream, an eye drop, or a salve.

### Materials and Methods

### Reagents and Antibodies

GMI, dissolved in PBS, was obtained from MycoMagic Biotechnology Co., Ltd. (New Taipei, Taiwan)(Yeh et al, 2022). TMZ was purchased from Merck Sharp & Dohme (Turku, Finland). Proteasome inhibitor (MG132) and lysosome inhibitor (Chloroquine) were purchased from Calbiochem (La Jolla, CA) and Enzo Life Sciences, Inc. (Farmingdale, NY), respectively. HGFR inhibitor, c-Met inhibitor 1 (HY), was purchased from MedChemExpress (Monmouth Junction, NJ).

Cycloheximide (ribosome inhibitor) and anti-pERK1/2 antibody were purchased from Sigma-Aldrich Chemical Co (St. Louis, MO). Antibodies against pAKT (S473) and pFAK (Y397) were purchased from Santa Cruz Biotechnology (Santa Cruz, CA). Antibodies against PARP, Caspase3, Slug, pHGFR (Y1234/Y1235), and HGFR were purchased from Cell Signaling (Beverly, MA). Antibodies against pSrc (Y418), Rad51, APNG, MGMT, and tubulin were purchased from GeneTex, Inc. (Hsinchu, Taiwan).

### Synergistic Analysis

Following the in vitro combined treatment, the data form each individual experiment were normalized to the un-treated (control) group and expressed as the percentage of cell viability. The inhibition rates of the combined treatment were analyzed using the CompuSyn™ program (Biosoft, USA). The program calculated a combination index (CI), which was used to identify the interaction of the tested drugs (GMI and TMZ) as synergistic (CI<1).

### Orthotopic Rat C6 Glioma-Bearing Rat Model

Adult female Sprague-Dawley rats were purchased from National Laboratory Animal Center (Taipei, Taiwan) and maintained according to the guidelines established in the Guide for the Care and Use of Laboratory Animals of the Institute of Laboratory Animal Resources Commission. The animal study was reviewed and approved by the Institutional Animal Care and Use Committee of Taipei Veterans General Hospital (Approved NO: VGH IACUC 2019-077).

The C6 cells were implanted while the rats were under general isoflurane anesthesia and immobilized on a stereotaxic unit (David Kopf Instruments, Tajunga, USA). The suspension of C6 cells (5×10⁵/15 μl) was implanted on day 1 into the left hippocampus (3 mm anterior and 2 mm lateral to bregma at a depth of 4 mm from the skull surface) with a flat Hamilton syringe (26G: Reno, Nevada) and a flow rate of 2 ml/min. The syringe was removed 5 minutes after injection to allow the cells to distribute into the left hippocampus and to minimize the diffusion of cells into unintended regions along the needle track as the needle was being removed. Rats were closely monitored daily for activity and behavior.

### In Vivo Magnetic Resonance Imaging (MRI)

All imaging experiments were performed using a Bruker 7T animal PET/MRI scanner with a 40 mm rat head volume coil. The main magnetic field was shimmed to minimize field inhomogeneity artifacts. Isoflurane (2-3%) gas was inhaled for anesthesia. After injection of the contrast agent Gadovist (0.03 mole/kg) into the tail vein, the administered animal was fixed on the animal bed in prone position and imaged with an axial and coronal T1W image sequence. The TIW parameters were as follows: Repetition time=325 ms, echo time=3.5 ms, field of view=35 mm×35 mm, matrix size=256×256, thickness=1 mm, the flip angle=60°, average=4, and the scan time=3 min 43 s. Tumor volume was determined by drawing irregular ROI to encircle the whole tumor in each image section-containing tumor using ImageJ software.

### Cell Lines and Cell Culture

The human Glioblastoma Multiforme GBM8401, U87, and rat GBM C6 cell lines were purchased from Bioresource Collection and Research Center (BCRC; Hsinchu, Taiwan). T98G cell lines were purchased from Japanese Cancer Research Resources Bank (JCRB; Sekisui, Korea). GBM8401 and C6 cells were cultured in Roswell Park Memorial Institute medium 1640 (RPMI medium 1640, GIBCO/Life Technologies) supplemented with 10% FBS. T98G cells were culture in Eagle's minimal essential medium (MEM, GIBCO/Life Technologies) with non-essential amino acids (NEAA, Thermo Fisher Scientific) and 10% FBS (VWR International LLC, Singapore). Adherent cells were detached by incubation with trypsin-EDTA (Invitrogen, Co., Carlsbad, CA). All cells were maintained at 37° C. in a humidified atmosphere of 5% CO₂.

### Cell Viability Assay

MTT (3-(4,5-dimethylthiazole-2-yl)-2,5-diphenyl tetrazolium bromide) assay was used to quantify cell viability. Cells were seeded in octuplicate on 96-well plates and incubated overnight prior to treatment. The treatments consisted of applying various concentrations of GMI (0-1.2 μM) for 24-72 h. The measured of cell viability as previously described (Lin et al, 2021).

### Western Blot Analysis

Cell extracts (30 μg) were then subjected to one-dimensional SDS-PAGE (8-12% separating gel) electrophoresis and were transferred to a PVDF membrane (0.22 μm, Millipore) by using a transfer apparatus (Bio-Rad, Richmond, CA). Membranes were blocked with 5% BSA in TBST buffer containing 0.05% Tween20. The expressions of proteins were identified by indicated antibodies. The expression of tubulin as an internal control. The indicated bands on the PVDF membrane were quantified using densitometry analysis by ImageJ software (National Institute of Mental Health, Bethesda, MD) and compared to results of the control group.

### In Vitro Transwell® Assays for Cell Migration and Invasion

Cells were pre-treated with GMI (0-0.6 μM) and seeded (5×10⁴ cells in 200 μl basal medium supplemented with 0.5% FBS) onto Transwell® chambers with/without matrix gel for 24 h. A staining assay using Liu's stain method was then performed (Lin & Hsu, (2016) Ling Zhi-8 reduces lung cancer mobility and metastasis through disruption of focal adhesion and induction of MDM2-mediated Slug degradation. Cancer Lett 375: 340-348).

### Cell Wound Closure Assay

Cells were seeded in 12-well plates and starved for 12 h. A scratch wound was then introduced into each well and washed with medium. The cells were then cultured in a culture medium with and without GMI (0-0.6 μM) for specific durations (0, 24 and 48 h). Wound closure was monitored using an optical microscope and measured using ImageJ software.

### Plasmids and Transfection

The pLKO.1_shLuc plasmid and Slug shRNA plasmids (Target Sequence of TRCN0000271239: CCGAAGCCAAATGACAAATAA and Target Sequence of TRCN0000284362: GAGTGACGCAATCAATGTTTA) were purchased from the National RNAi Core Facility of Taiwan for gene silencing and were verified by sequencing.

### Lentivirus Package

Slug-shRNA containing Lentivirus was produced in HEK293T cells that were transfected with Slug-shRNA plasmid, virus packaging genes plasmid and envelope genes plasmid using Lipofectamine 2000 and following the manufacturer's protocol. The media containing lentiviruses from the HEK293T cells was harvested for 40 and 64 h. GBM8401 cells were infected with the lentiviruses and selected for experiments using puromycin (Sigma-Aldrich).

The Quantitative Reverse Transcription Polymerase Chain Reaction (qRT-PCR)

Total RNA from cells was extracted using a Quick-RNA Miniprep Kit (Zymo Research, Irvine, USA). 1 μg of total RNA was reverse transcribed using a PrimeScript™ RT reagent Kit (Takara, Shiga, Japan). The cDNA product was produced using a TB Green® Premix Ex Taq II (Tli RNaseH Plus, Takara, Shiga, Japan) on StepOnePlus™ Real-Time PCR System (Applied Biosystems, Foster City, USA). The primers that were used for qRT-PCR were Slug-F: ATGCATATTCGGACCCACACATTAC, Slug-R: AGATTTGACCTGTCTGCAAATGCTC, and GAPDH-F: GCTACACTGAGGACCAGGTTGTC and GAPDH-R: AGCCGTATTCATTGTCATACCAGG. Data was quantitated by 2^{-ΔCt} (ΔCt=Ct of target gene−Ct of GAPDH; Ct: cycle threshold).

### Apoptosis Analysis

Briefly, cells (2.5×10⁵ cells/plate) were treated with GMI and TMZ (50 μM) for 48 h. Following treatment, the cells were harvest and stained by FITC Annexin

V/Dead Cell Apoptosis Kit (Invitrogen). Samples were determined by flow cytometry (BD FACSCalibur), and raw data was analyzed by FlowJo software for cell apoptosis analysis.

### Histology and Immunohistochemical Staining

Tumor cryosections (20 μm) were blocked with 2% normal donkey serum (Jackson Immuno-Research) and incubated with one of the following primary antibodies: rabbit anti-HGFR antibody (1:500; Abcam) and mouse anti-his tag (GMI) antibody (1:500; Millipore). The appropriate secondary antibodies and the avidin-biotin-peroxidase complex (Vectastain Elite ABC kit: Vector Laboratories Inc., USA), with appropriate chromogens, were used to visualize the antibodies and examined by Leica confocal microscopy. The intensity of staining was evaluated by choosing the slice with the largest tumor area in the magnification of 200× and obtaining the average intensity of staining from four areas (1388*1040 pixel) in this slice using Image J software.

### Tumor Volume, Tumor Histology, and Animal Survival

Five days after tumor implantation surgery, rats were randomly assigned into four group (6 animals/group) to be treated with different drugs every other day for 4 times, i.e. on day 5, 7, 9, 11.
Group 1 (control): intravenous administration of saline.
Group 2 (GMI): intravenous (iv) administration of GMI (0.6 mg/Kg)
Group 3 (TMZ): intraperitoneal (ip) administration of TMZ (30 mg/Kg)
Group 4 (GMI+TMZ): combination of GMI (iv, 0.6 mg/Kg) and TMZ (ip, 30 mg/Kg)

MRI imaging acquisition was performed on day 13 and a half of the animals in each group were sacrificed on day 15 for immunohistochemistry study. After tumor volume was measured, specimens from the animals in the histology group were subjected to other histological evaluations described as follows. The other animals were observed to for survival. If the animals displayed clinical signs of suffering including anorexia or more than 20% of weight loss, sacrifice would be performed for ethical reasons.

### Statistical Analysis

Data were presented as means±Standard deviation (SD). In bar graphs, SD values were indicated with error bars. Samples were compared statistically using Graphpad Prism8. Statistical analyses were performed by Student's unpaired t tests when applicable. P values of less than 0.05 were considered to be significant.

### Examples

Exemplary embodiments of the present disclosure are further described in the following examples, which should not be construed to limit the scope of the present disclosure.

### Example 1. GMI Inhibited Cell Viability and Induced Apoptosis in GBM Cells

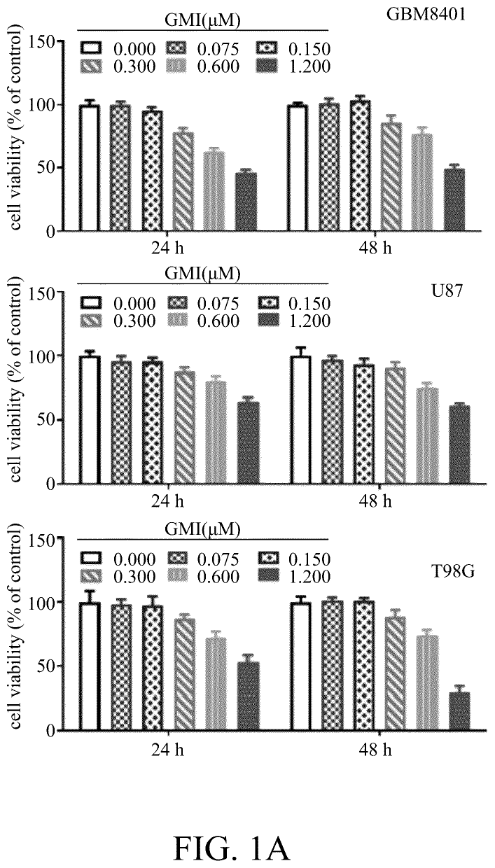

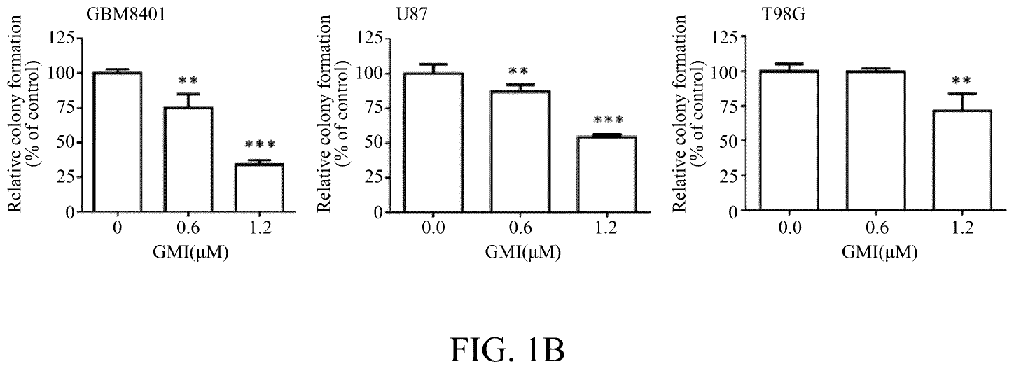

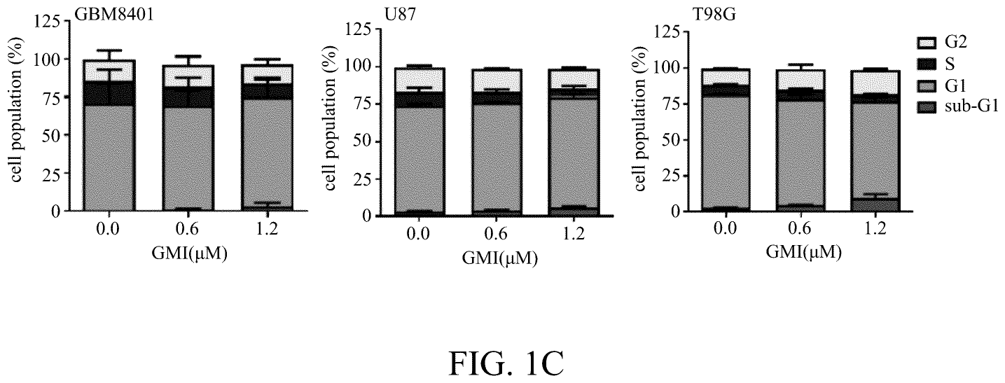

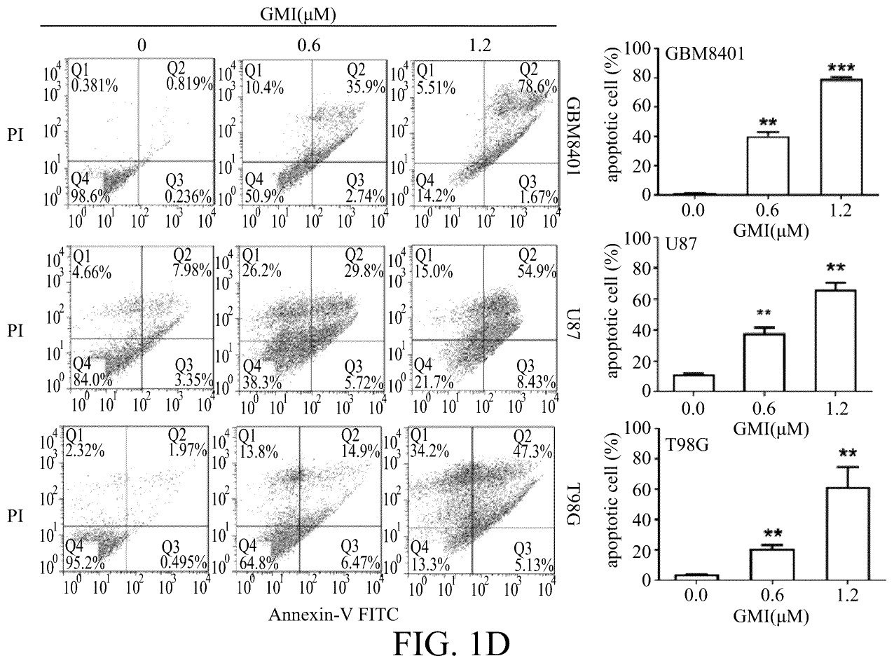

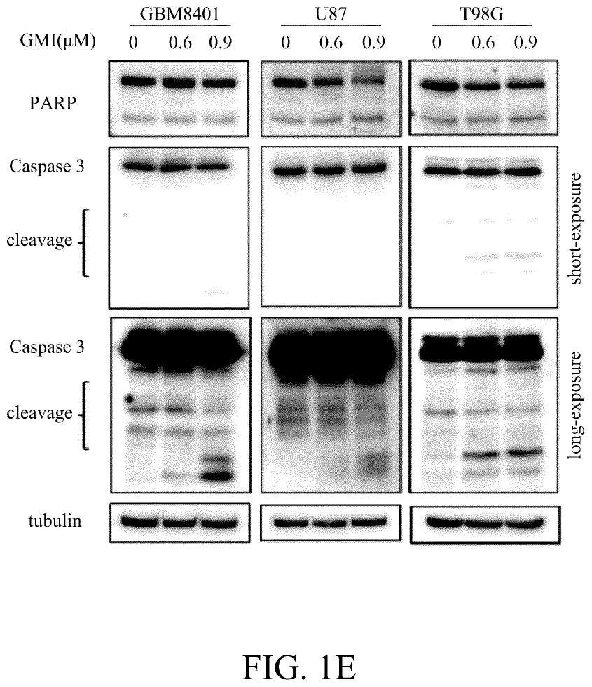

The human brain glioblastoma multiforme GBM8401, U87 and T98G were chosen to investigate the anti-GBM cell activity of GMI. The data were representative of three separate experiments and were presented as the means±SD; error bars indicated SD. Significant differences are shown (*p<0.05 compared to the control group). The apoptosis on the tested cells were analyzed by FlowJo software. As shown in FIG. 1A, GMI effectively inhibited cell viability of GBM cells. The IC₅₀ of GMI in those GBM cells were further determined using the CompuSyn™ software. The IC₅₀ of GMI in these GBM cells were lower than 5 μM. Specifically, the IC₅₀ of GMI in GBM8401, U87 and T98G cells were about 1.41, 2.08 and 1.23 μM, respectively. In addition, GMI effectively inhibited colony formation in a dose-dependent manner (shown in FIG. 1B). GMI induced a sub-G1 population and significantly promoted apoptosis (shown in FIGS. 1C-1D). Moreover, GMI increased the expressions of cleavage forms of caspase 3 and PARP (shown in FIG. 1E). Taken together, the results showed that GMI suppressed GBM cells may depend on induction of apoptosis.

### Example 2. GMI Inhibits Tumor Growth and Prolongs Survival Rate in an Orthotopic Rat Model of Glioblastoma

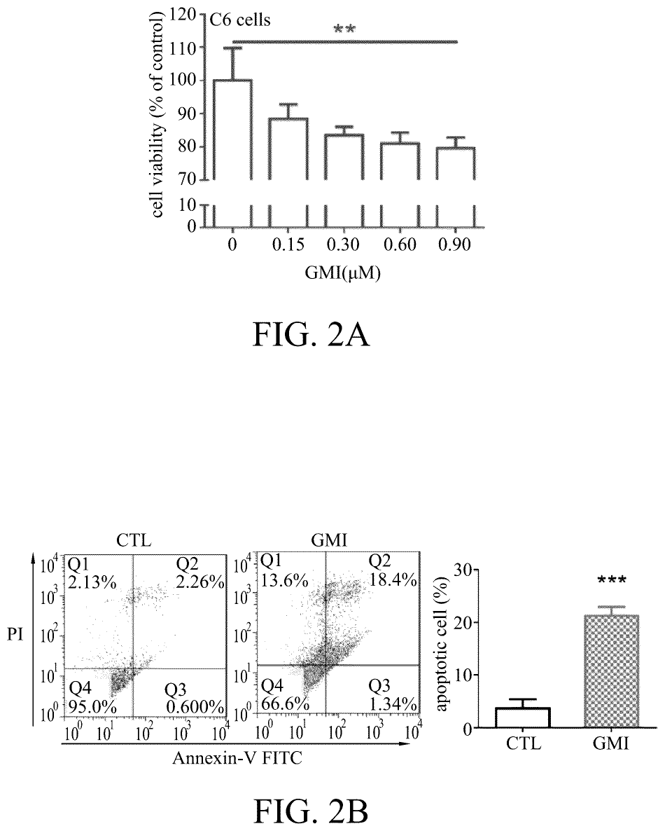

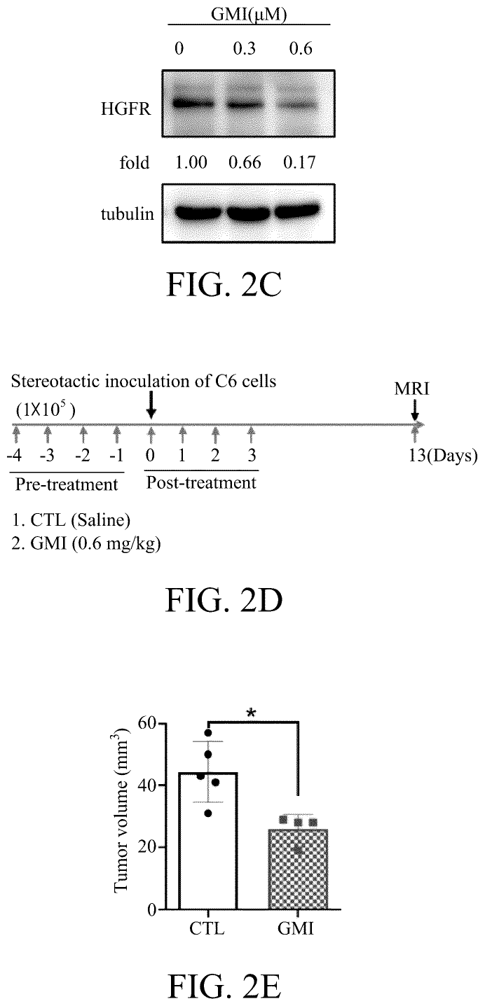

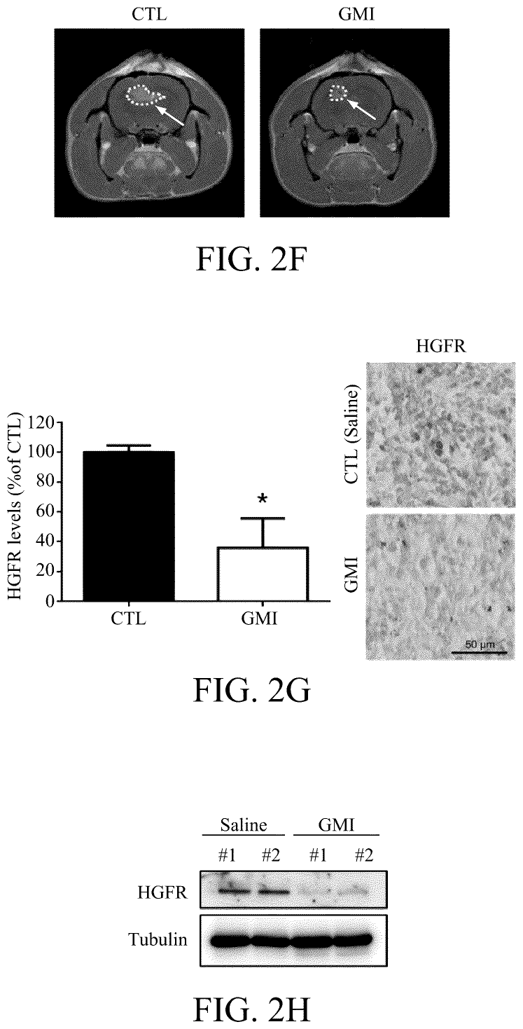

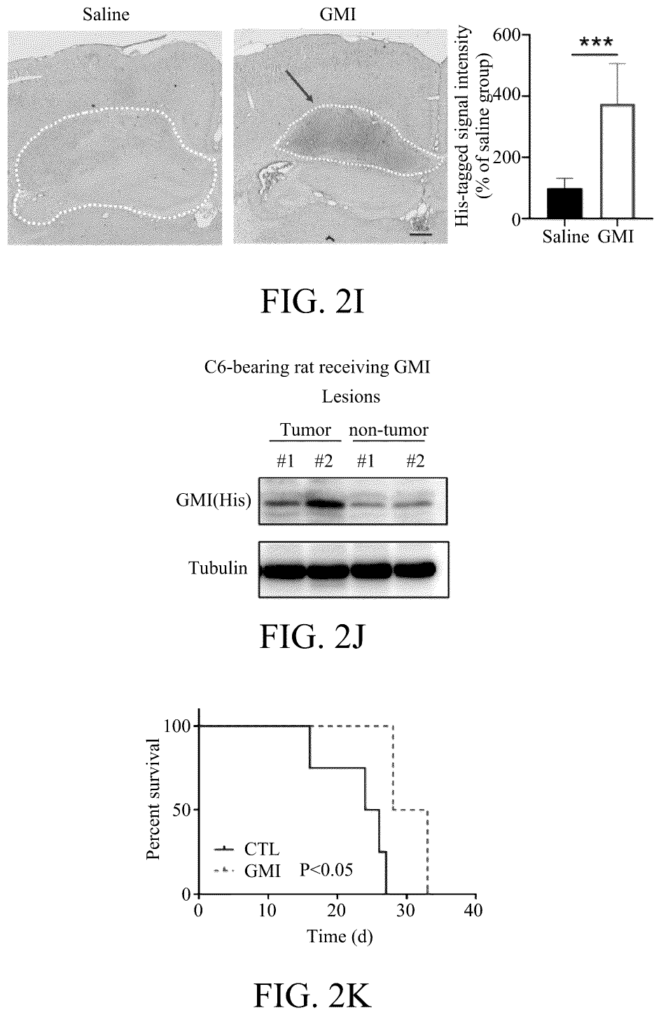

To examine the anti-tumor efficacy of GMI, we developed an orthotopic rat model to examine the therapeutic effects of GMI on a Rattus Norvegicus brain glioma C6 cells-bearing rat model in vivo. Significant differences are shown (*p<0.05 compared to the control group). Initially, we showed that GMI inhibited cell viability of C6 cells but increased the population of apoptosis in vitro (FIGS. 2A and 2B). Also, GMI downregulated expression of HGFR (FIG. 2C). These results suggested that GMI could inhibit GBM C6 cells. Next, we examined the efficacy of tail-vein injected GMI for the entire experimental period (FIG. 2D). Tumor volumes of rats were significantly reduced compared with the rats that received saline (CTL group; FIGS. 2E and 2F). We further detected the HGFR expression in GBM tissue by immunohistochemistry and Western blot assay. As shown in FIGS. 2G and 2H, HGFR levels were significantly decreased in GBM tissues of rats that received GMI. Importantly, we found that GMI was located in the tumor lesion (shown in FIG. 2I). Compared to non-tumor lesions, we measured the amount of GMI detected in tumor lesions of C6-bearing rats receiving GMI (shown in FIG. 2J). Furthermore, continuous injection with GMI exhibited more efficacy in prolonging the survival rate compared with that of the control group (shown in FIG. 2K). Together, these results indicate that continuous vein-injection with GMI elicits the greatest efficacy in suppressing tumorigenesis.

### Example 3. GMI Inhibits GBM Patients-Derived Cells

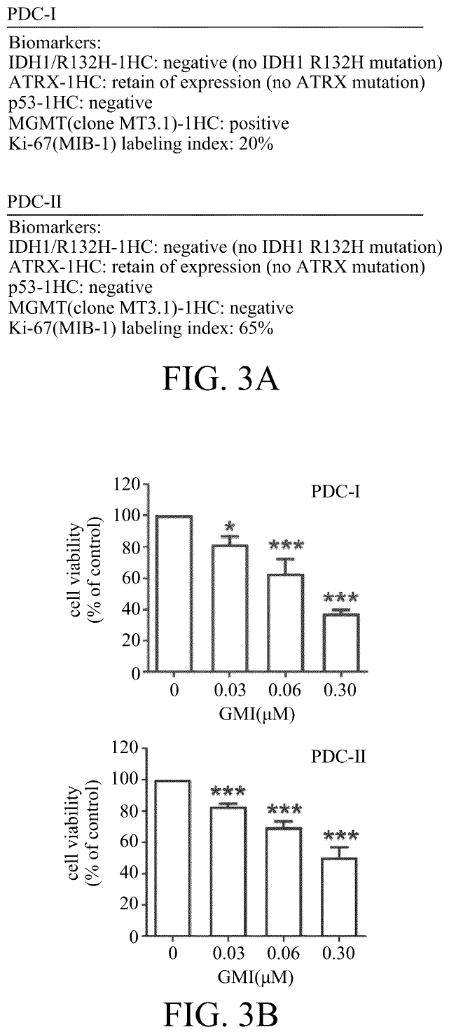

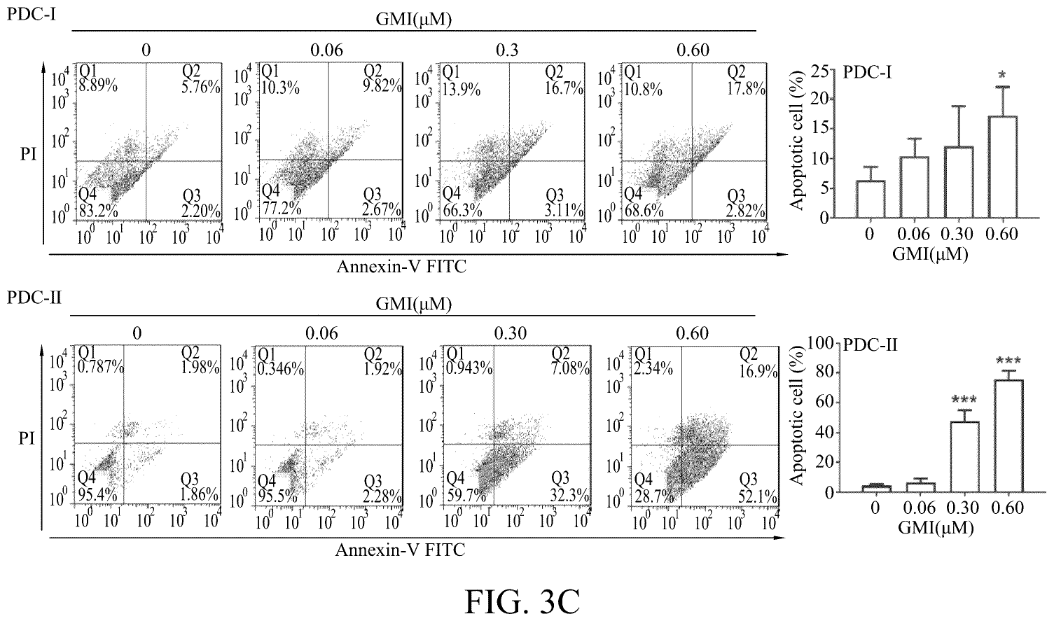

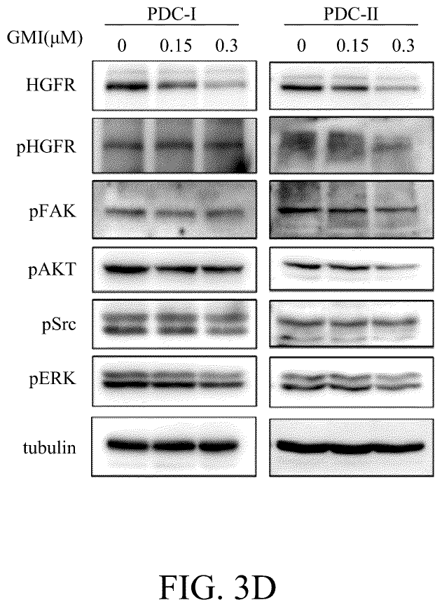

To extend our findings from GBM cells, we evaluated the antitumor activity of GMI and associated mechanistic endpoints in two GBM patients-derived cells (PDC-I and PDC-II; FIG. 3A). We found that GMI concentration-dependently inhibited cell viability and reduced the number of both PDCs (shown in FIG. 3B). Analysis of cell death revealed that GMI significantly induced apoptosis in a concentration-dependent manner (shown in FIG. 3C). In these PDCs, GMI treatment resulted in decreased expression of HGFR levels, as well as inhibition of HGFR-related signal molecules such as pHGFR, pFAK, pSrc, pAKT and pERK (shown in FIG. 3D). These results are consistent with the results observed in various GBM cell lines. Together, these findings suggested that GMI displays anti-GBM activity.

### Example 4 and Example 5. GMI Enhanced Synergistically Temozolomide (TMZ)-Induced Cytotoxic Effects

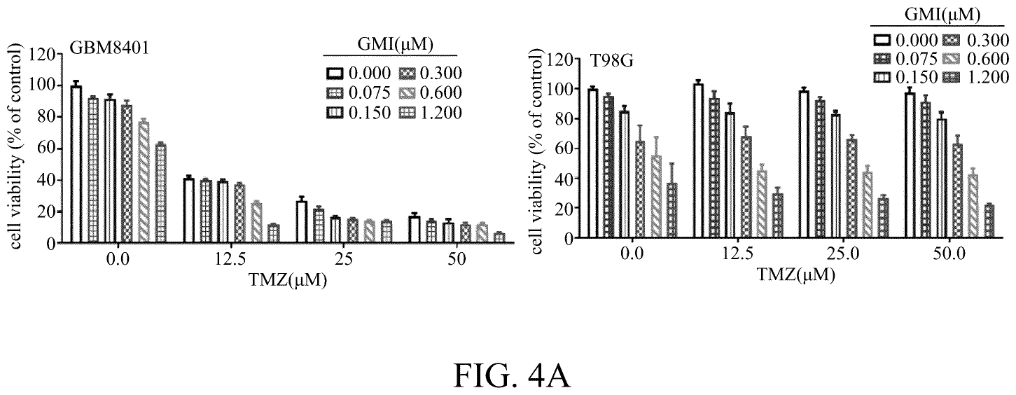

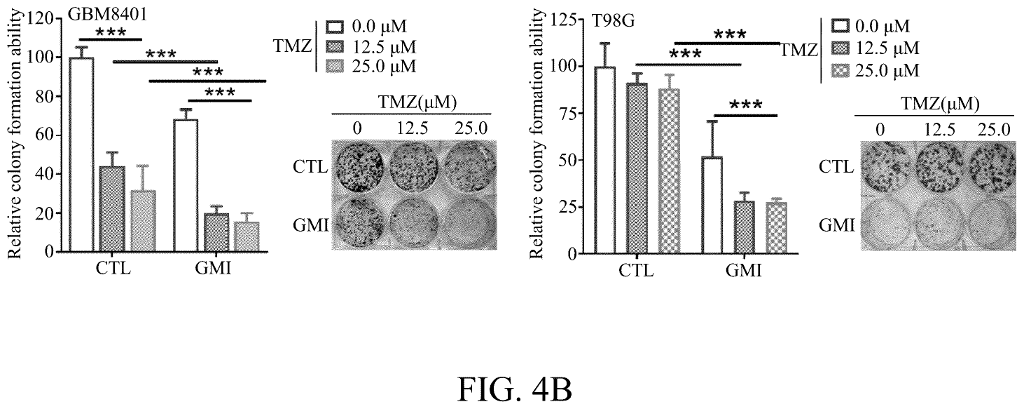

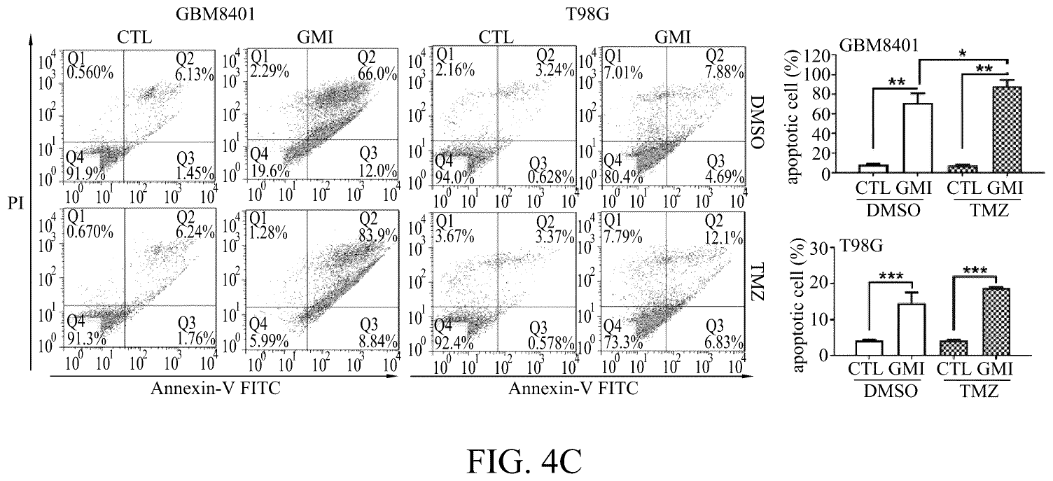

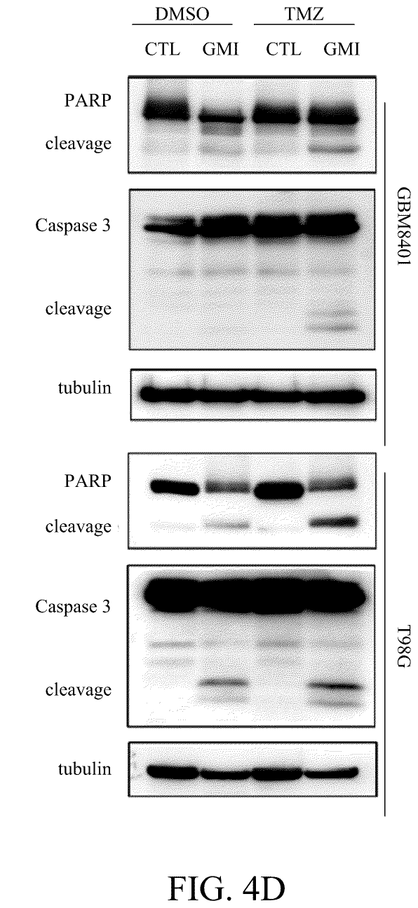

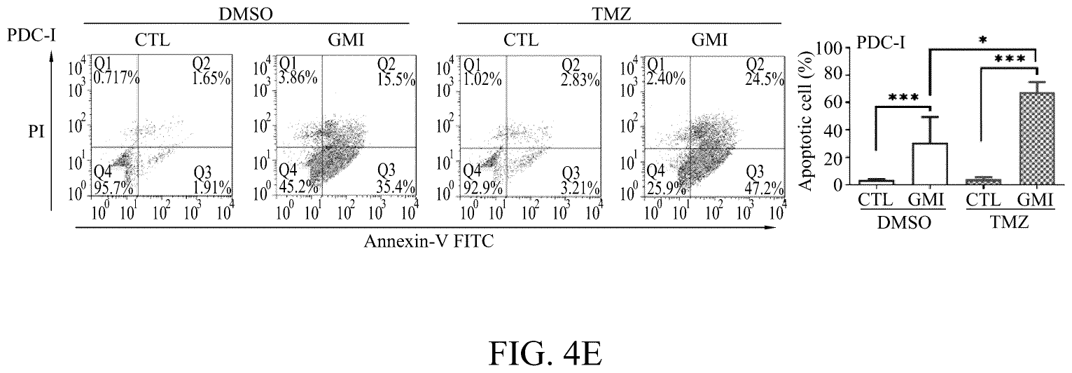

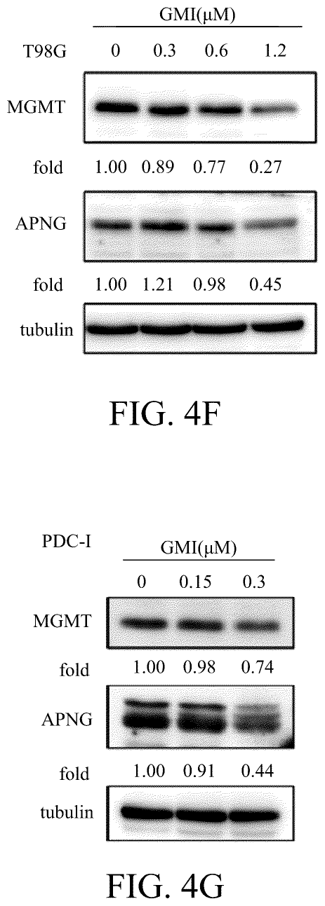

GBM8401 and T98G, which are TMZ-responsive and -resistant GBM cells, were chosen to evaluate the efficacy of GMI combined with TMZ. Analysis of cell viability and colony formation revealed that the combination of TMZ (0-50 μM) with GMI (0-1.2 μM) presented a greater inhibitory effect than individual treatment (shown in FIGS. 4A-4B). The synergistic effects (combination index; CI values (Chou & Talalay, (1984) Quantitative analysis of dose-effect relationships: the combined effects of multiple drugs or enzyme inhibitors. Adv Enzyme Regul 22: 27-55)) of GMI combined with TMZ on the GBM cells are shown in Table 1. Specifically, GMI combined with TMZ induced a synergistic growth-inhibitory effect (CI<1.0). Higher efficacy of the combined strategy was shown in T98G cells. In addition, we evaluated the dose-reduction index (DRI) (Chou, (2006) Theoretical basis, experimental design, and computerized simulation of synergism and antagonism in drug combination studies. Pharmacol Rev 58: 621-681: Lin et al, (2019) Sodium thiosulfate enhances production of polysaccharides and anticancer activities of sulfated polysaccharides in Antrodia cinnamomea. Carbohydr Polym 216: 204-212) in the combined treatment of testing cells and found most of the DRI of combined treatment in GBM8401 and T98G cells exceeded one (Table 1), suggesting that GMI may decrease the effective dosage amount of TMZ.

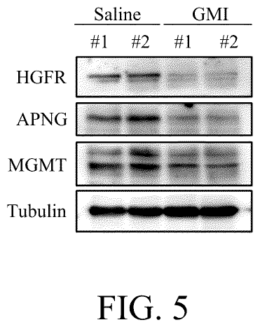

Thus, we further examined the role of GMI in TMZ-induced apoptotic response. As expected, we found that GMI-combined with TMZ treatment in GBM8401 cells and T98G cells induced enhanced apoptotic responses compared to that from using either GMI or TMZ treatment individually (show in FIG. 4C). Western blotting analysis revealed that GMI effectively increased the cleavage forms of caspase 3 and PARP in TMZ-treated cancer cells (shown in FIG. 4D). In addition, we found that the combined strategy significantly induced apoptotic responses in TMZ non-sensitive PDC-I cells (shown in FIG. 4E). We further found that GMI downregulated alkylpurine-DNA-N-glycosylase (APNG) and 06-methylguanine methyltrasferase (MGMT) levels in T98G cells, PDC-I and tumor lesions of rats receiving GMI (shown in FIGS. 4F-4G and FIG. 5). Evidence shows that MGMT and APNG are two key molecules which are correlated to TMZ-resistance. Taken together, GMI may be suggested as a supplemental agent for enhancing the efficacy of TMZ in GBM treatment via downregulation of MGMT and APNG.

TABLE 1

| | | GBM8401 | | | | T98G | | | |
|---|---|---|---|---|---|---|---|---|---|
| | | Inhibition | | | | Inhibition | | | |
| Concentrations | | of cell | | | | of cell | | | |
| GMI | TMZ | viability | | DRI | | viability | | DRI | |
| (μM) | (μM) | (%) | CI | GMI | TMZ | (%) | CI | GMI | TMZ |
| 0.075 | 12.5 | 60 | 0.97 | 59.26 | 1.04 | 6.2 | 0.05 | 21.94 | 61564 |
| | 25 | 78 | 0.7 | 181.25 | 1.43 | 7.9 | 0.02 | 57.98 | 131873 |
| | 50 | 86 | 0.78 | 348.91 | 1.29 | 8.7 | 0.01 | 113.3 | 179814 |
| 0.15 | 12.5 | 60 | 0.97 | 30.43 | 10.7 | 15.7 | 1.14 | 0.88 | 1405.37 |
| | 25 | 83 | 0.48 | 139.3 | 2.11 | 17.1 | 1.01 | 0.99 | 839.53 |
| | 50 | 87 | 0.7 | 196.38 | 1.44 | 20.0 | 0.81 | 1.24 | 586.72 |
| 0.3 | 12.5 | 63 | 0.9 | 17.13 | 1.19 | 31.9 | 0.78 | 1.29 | 7005.39 |
| | 25 | 85 | 0.44 | 78.06 | 2.34 | 34.0 | 0.7 | 1.44 | 4133.48 |
| | 50 | 88 | 0.63 | 78.06 | 1.62 | 36.8 | 0.6 | 1.66 | 2558.19 |
| 0.6 | 12.5 | 75 | 0.49 | 111.5 | 2.29 | 54.7 | 0.52 | 1.93 | 36447.3 |
| | 25 | 86 | 0.41 | 17.67 | 2.56 | 55.8 | 0.49 | 2.04 | 19691.9 |
| | 50 | 88 | 0.63 | 43.16 | 1.63 | 57.5 | 0.45 | 2.21 | 11107.7 |
| 1.2 | 12.5 | 88 | 0.18 | 56.43 | 6.75 | 70.1 | 0.48 | 2.09 | 115751 |
| | 25 | 86 | 0.42 | 29.27 | 2.64 | 73.6 | 0.39 | 2.56 | 78255 |
| | 50 | 94 | 0.29 | 22.27 | 3.62 | 77.9 | 0.3 | 3.36 | 58871.2 |

### Example 6 and Example 7. Combination of GMI and TMZ Suppresses Tumor Growth in Orthotopic C6 Cell-Bearing Rats

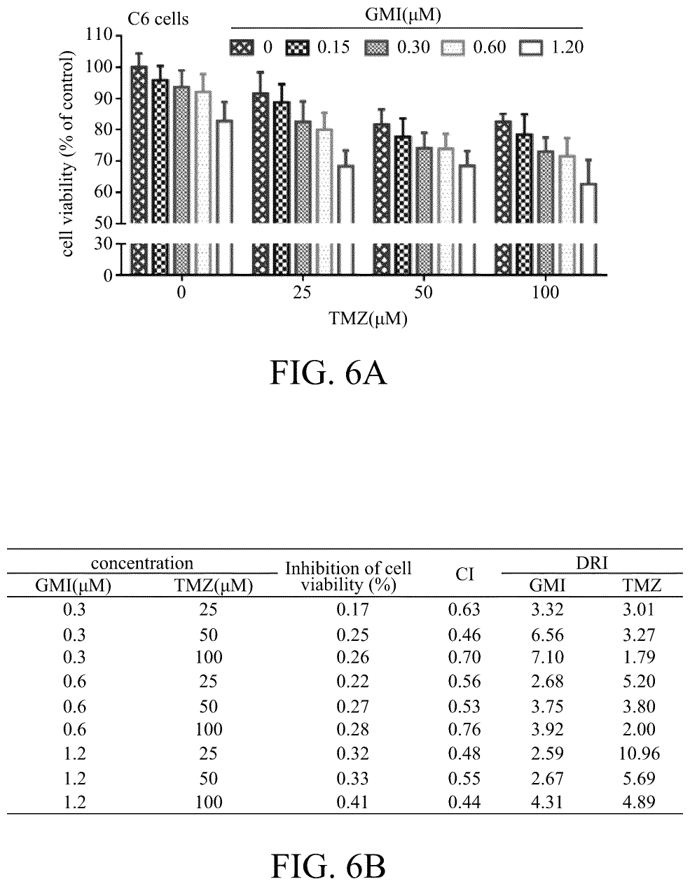

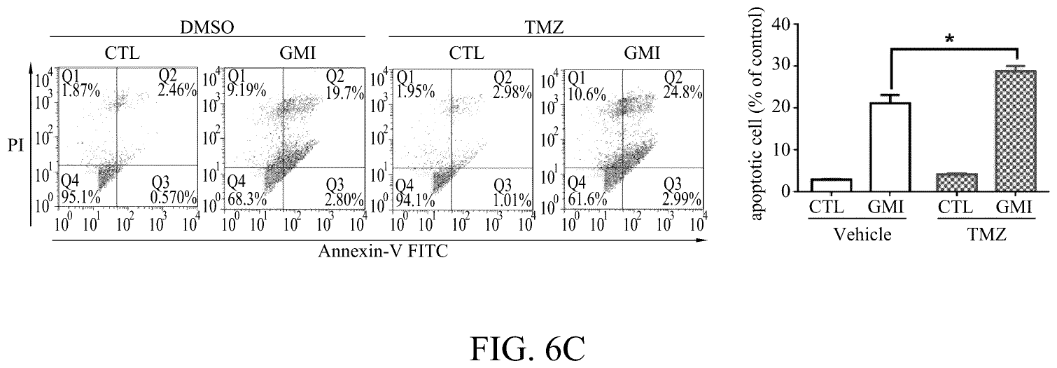

To examine the efficacy of GMI combined with TMZ in orthotopic C6 cell-bearing rats, we initially examined the cell viability of C6 cells upon GMI and TMZ co-treatment. As expected, GMI combined with TMZ effectively inhibited cell viability of C6 cells ((shown in FIG. 6A). Specifically, GMI combined with TMZ exhibited the high efficacy of the synergistic growth-inhibitory effect (CI<1.0) and DRI values (DRI>1.0; FIG. 6B). Moreover, GMI-combined with TMZ treatment in C6 cells significantly induced apoptosis compared to that using GMI treatment alone (shown in FIG. 6C).

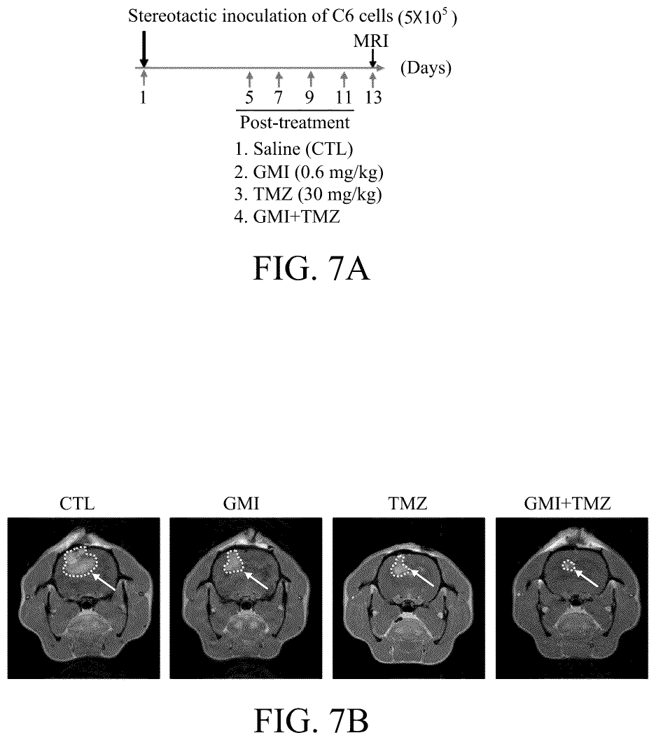

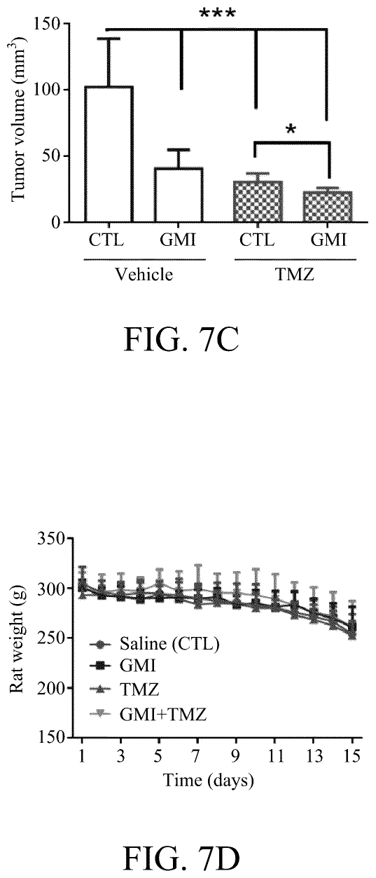

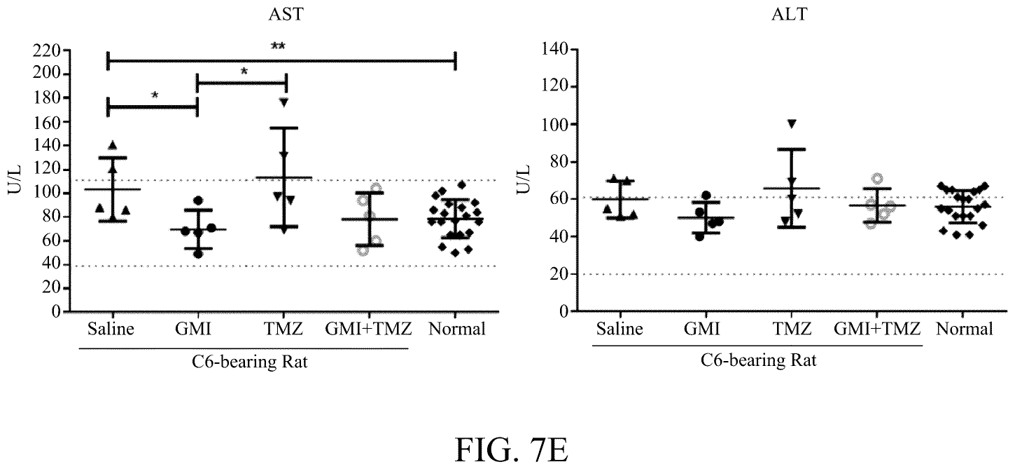

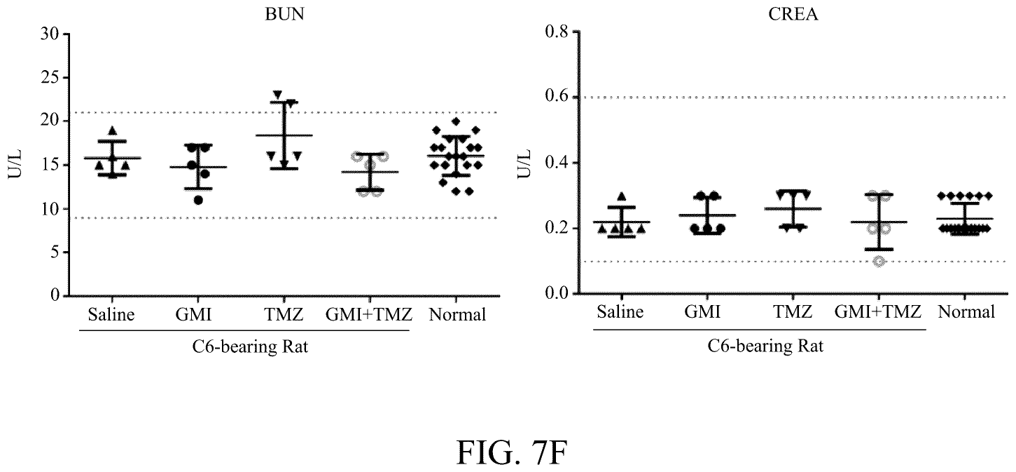

Next, we examined the effects of GMI combined with TMZ on GBM tumor growth of C6-cell bearing rats (shown in FIG. 7A). We found that compared with individual treatments with GMI and TMZ, the combination of GMI and TMZ was more effective at suppressing brain tumor volume (shown in FIGS. 7B-7C). We also monitored the cytotoxic effects of the combination strategy. No apparent adverse effects on rat body weight were observed with the indicated treatment compared to the control group (shown in FIG. 7D). Rats injected with C6 cells and treated with TMZ increased AST/ALT expression; whereas, GMI effectively reduced the values of AST/ALT compared to the normal baseline (shown in FIG. 7E). In addition, rats injected with TMZ showed slightly increased BUN expression: however, no difference in each experimental group was observed (shown in FIG. 7F). These results suggest that GMI might eliminate the side effects of TMZ, while enhancing the efficacy of GBM suppression.

### Example 8. GMI Inhibited Cell Migration and Invasion of GBM8401 Cells

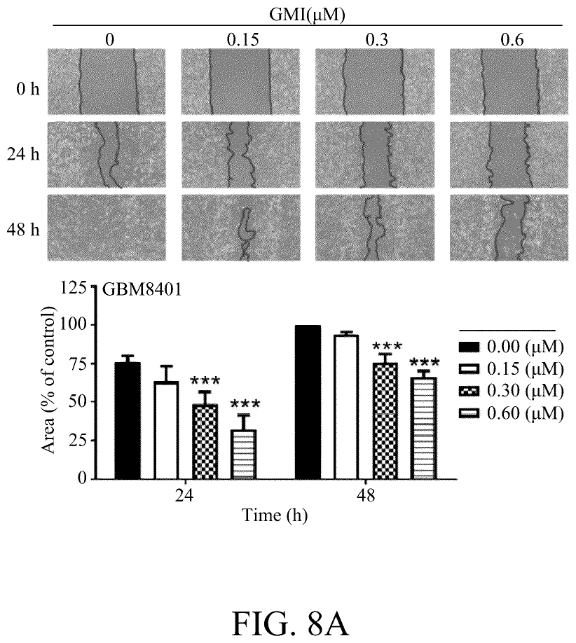

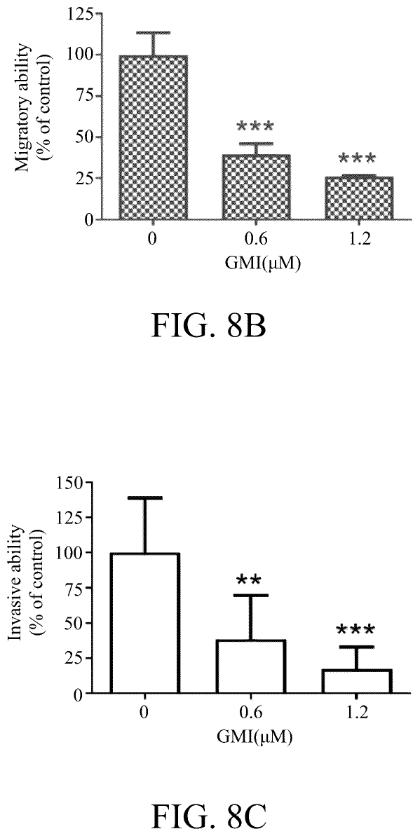

We examined the effect of GMI on the mobility of GBM8401 cells. Cell migration was initially quantified in vitro using a wound-healing assay. Data is representative of three independent experiments and is presented as mean±SD. SD is indicated by error bars. P values are considered to be significant at the level of 0.01**. As shown in FIG. 8A, the healing rates were reduced by GMI in time and dose-dependent manners. A Transwell® assay was used to confirm the ability of GMI to inhibit the migration and invasion of GBM8401 cells (FIGS. 8B and 8C). The number of migratory and invasive cells was decreased by more than 50%, compared to the control group, following treatment with GMI (1.2 μM). These results showed that GMI inhibited the migration and invasion of GBM8401 cells.

### Example 9. GMI Combined with TMZ Inhibited Cell Mobility

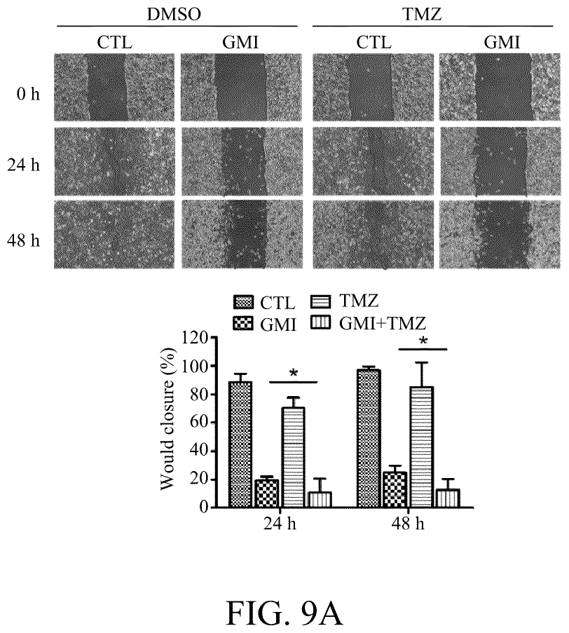

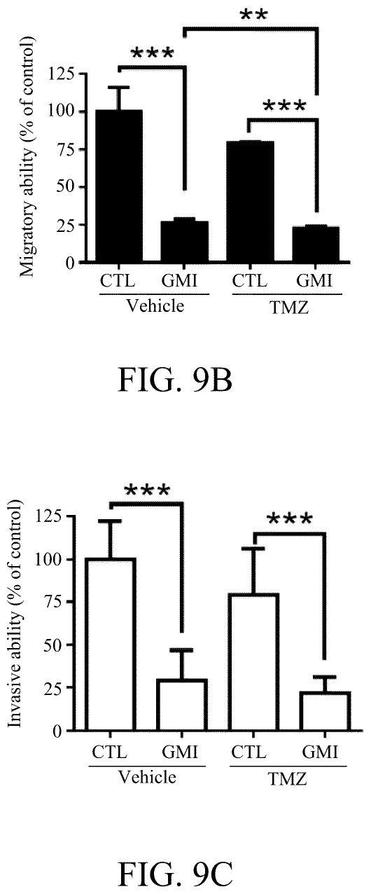

We examined the effect of GMI combined with TMZ on the migratory and invasive properties of GBM8401 cells. Data is representative of three independent experiments and is presented as mean±SD. SD is indicated by error bars. P values were considered significant at the level of 0.05*. The results of an in vitro wound-healing assay showed that TMZ (50 μM) did not inhibit cell migration, but a high dosage of GMI (1.2 μM) significantly suppressed cell migration (shown in FIG. 9A). The combined treatment significantly reduced the healing rate in a time-dependent manner (shown in FIG. 9A), so a combination of GMI and TMZ inhibits cell migration more than GMI treatment only. Furthermore, a Transwell®) assay was used to determine how GMI combined with TMZ inhibited the mobility of GBM8401 cells (shown in FIGS. 9B and 9C). As expect, TMZ slightly reduced migration and invasion. Significantly, there are 50% fewer migratory and invasive cells compared to control group following co-treatment with GMI (1.2 μM) and TMZ (50 μM). These results showed that GMI may be used as an element of an anti-mobility strategy for TMZ therapy.

### Example 10. GMI Induced Proteasome-Dependent Slug Degradation

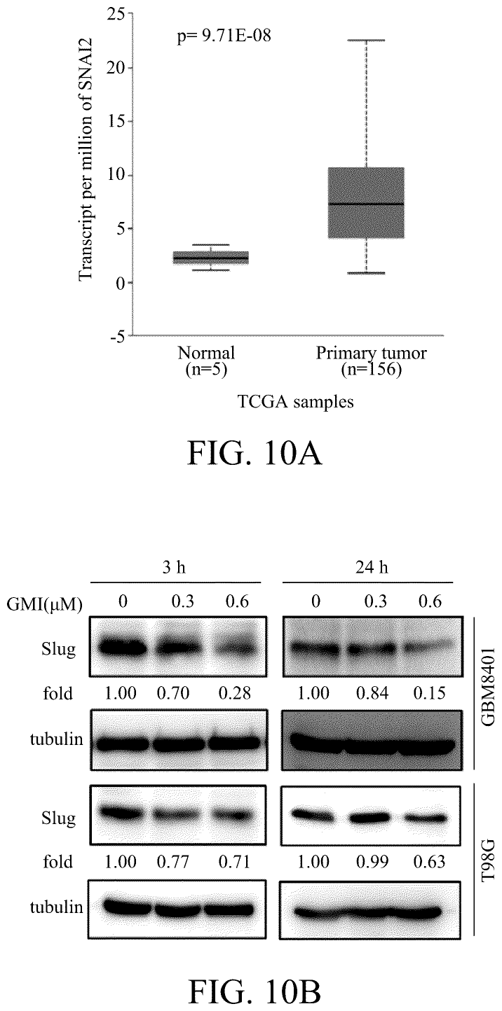

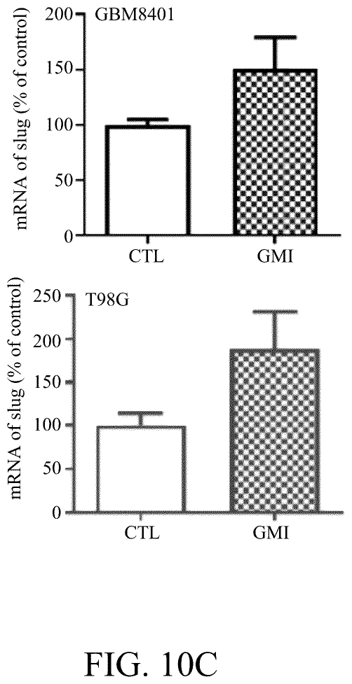

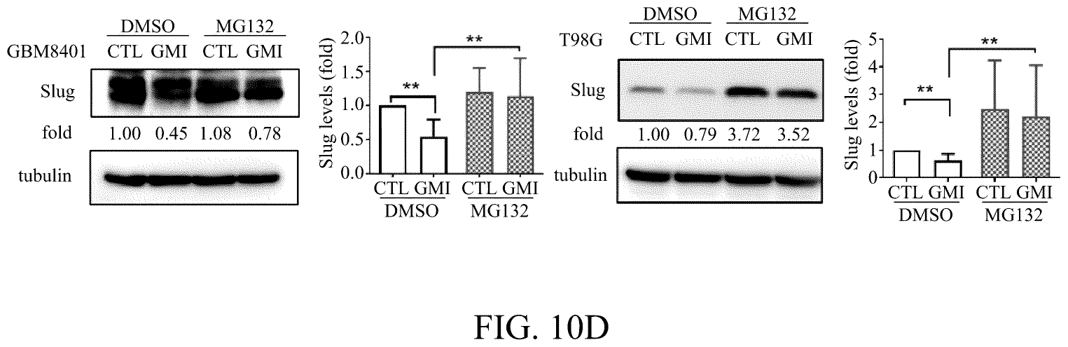

To determine the role of Slug in patients with GBM, the Cancer Genome Atlas (TCGA) database was used to measure the expression of Slug in relation to GBM. Western blot analysis was performed to determine the levels of Slug. Tubulin was used as the internal control. The intensity of the bands of Slug in experiments was measured using ImageJ. Data is representative of three separate experiments and is presented as mean±SD. SD is indicated by error bars. P values are considered to be significant at the level of 0.01**. We found that the transcript of SNAI2 significantly increased in the primary GBM group, compared to the normal group (FIG. 10A). Therefore, the role of Slug in GMI-treated GBM cells was studied by measuring the expression of Slug in GBM8401 and T98G cells after GMI treatment. Initially, we found that GMI inhibited the expression of Slug during short and long-term treatment (shown in FIG. 10B). However, GMI increased mRNA levels of Slug (shown in FIG. 10C). These results showed that GMI mediated protein stability for Slug. Using MG132 to inhibit proteasome activity, the results of this study showed that MG132 counteracted GMI-induced Slug degradation (shown in FIG. 10D). These results showed that GMI could induce the degradation of Slug in GBM cells.

1. A method for treating brain tumor, comprising administering a subject in need thereof an effective amount of an immunomodulatory protein of Ganoderma, a recombinant thereof, or a fungal immunomodulatory protein of a similar structure.

2. The method of claim 1, wherein the brain tumor is a refractory brain tumor.

3. The method of claim 1, wherein the brain tumor is a high-grade glioma.

4. The method of claim 3, wherein the high-grade glioma is a glioblastoma multiforme (GBM).

5. The method of claim 4, wherein the GBM is a refractory GBM.

6. The method of claim 5, wherein the refractory GBM is refractory to temozolomide (TMZ).

7. The method of claim 1, wherein the treatment of the brain tumor comprises suppressing hepatocyte growth factor receptor (HGFR), O⁶-methylguanine methyltrasferase (MGMT) or alkylpurine-DNA-N-glycosylase (APNG) expression, and/or inducing proteasome-dependent Slug degradation in a brain tumor.

8. The method of claim 1, wherein the treatment of the brain tumor comprises reducing growth, invasion, and/or metastasis of the brain tumor.

9. The method of claim 1, wherein the immunomodulatory protein of Ganoderma, the recombinant thereof, or the fungal immunomodulatory protein of a similar structure has:
an amino acid sequence of (1) TLAWNVK (SEQ ID NO: 1), (2) PNWGRGRPSSFIDT (SEQ ID NO: 2), and (3) YNSGYGIADTN (SEQ ID NO: 3);
an amino acid sequence of MSDTALIFTLAWNVKQLAFDYTPNWGRGRPSSFIDTVTFPTVLTDK AYTYRVVVSGKDLGVRPSYAVESDGSQKINFLEYNSGYGIADTNTIQVYVIDPDTGN NFIVAQWN (SEQ ID NO: 4); or
an amino acid sequence of
(SEQ ID NO: 5)
EAEAEFMSDTALIFTLAWNVKQLAFDYTPNWGRGRPSSFIDTVTFPTVL
TDKAYTYRVVVSGKDLGVRPSYAVESDGSQKINFLEYNSGYGIADTNTI
QVYVIDPDTGNNFIVAQWNYLEQKLISEEDLNSAVDHHHHHH.

10. The method of claim 1, wherein the Ganoderma is Ganoderma lucidum, Ganoderma tsugae, Ganoderma microsporum, Ganoderma applanatum, Ganoderma japonicum, Ganoderma astum, Ganoderma atrum, or Ganoderma sinensis.

11. The method of claim 1, wherein the immunomodulatory protein of Ganoderma, the recombinant thereof, or the fungal immunomodulatory protein of a similar structure is selected from the group consisting of LZ-8, FIP-gts, GMI, FIP-gap, FIP-gja, FIP-gas, FIP-gat, FIP-gsi, a recombinant LZ-8, a recombinant FIP-gts, a recombinant GMI, a recombinant FIP-gap, a recombinant FIP-gja, a recombinant FIP-gas, a recombinant FIP-gat, and a recombinant FIP-gsi.

12. The method of claim 1, wherein the immunomodulatory protein of Ganoderma, the recombinant thereof, or the fungal immunomodulatory protein of a similar structure is administered in combination with an anti-cancer agent.

13. The method of claim 12, wherein the anti-cancer agent is TMZ.

14. The method of claim 12, wherein the immunomodulatory protein of Ganoderma, the recombinant thereof, or the fungal immunomodulatory protein of a similar structure are administered with the anti-cancer agent simultaneously, sequentially, or separately.

15. The method of claim 12, wherein the immunomodulatory protein of Ganoderma, the recombinant thereof, or the fungal immunomodulatory protein of a similar structure is administered in combination with radiotherapy and/or chemotherapy.

16. The method of claim 1, wherein the immunomodulatory protein of Ganoderma, the recombinant thereof, or the fungal immunomodulatory protein of a similar structure is administered orally or rectally.

17. The method of claim 1, wherein the immunomodulatory protein of Ganoderma, the recombinant thereof, or the fungal immunomodulatory protein of a similar structure is administered parenterally.

18. The method of claim 1, wherein the effective amount of the immunomodulatory protein of Ganoderma, the recombinant thereof, or the fungal immunomodulatory protein of a similar structure is about 0.01 mg to about 100 mg protein per 70 kg human body weight.
